(19) United States
(12) Patent Application Publication
(10) Pub. No.: US 2008/0045504 A1
(43) Pub. Date: Feb. 21, 2008

(54) Benzonitrile Derivatives to Treat Musculoskeletal Frailty

(75) Inventors: Thomas G. Gant, Carlsbad, CA (US); Bruce A. Lefker, Gales Ferry, CT (US); Hua Zhu Ke, Newbury Park, CA (US); James P. O'Malley, Quaker Hill, CT (US); Roger J. Hill, Salem, CT (US)

Correspondence Address: PFIZER INC, 10555 SCIENCE CENTER DRIVE, SAN DIEGO, CA, 92121, US

(73) Assignee: PFIZER PRODUCTS INC., Groton, CT (US)

(21) Appl. No.: 11/568,887

(22) PCT Filed: Apr. 29, 2005
(86) PCT No.: PCT/IB05/01250

§ 371 Date: Nov. 9, 2006

Related U.S. Application Data
(60) Provisional application No. 60/569,874, filed on May 11, 2004.
(60) Provisional application No. 60/658,448, filed on Mar. 3, 2005.

(51) Int. Cl. A61K 31/277; A61K 31/40; A61K 31/451; A61K 31/55; A61P 19/00; A61P 21/00; C07C 255/58; C07D 207/08; C07D 211/06; C07D 295/14

(52) U.S. Cl. 514/217.12; 514/331; 514/429; 514/646; 540/610; 546/230; 548/566; 558/411

(57) ABSTRACT

This invention relates to novel amino substituted benzonitrile derivatives and to pharmaceutical compositions containing the novel amino substituted benzonitrile derivatives. This invention also relates to methods of treatment using amino substituted benzonitrile derivatives to prevent and/or restore age-related decline in muscle mass and strength, treat a wasting disease, treat a condition that prevents with low bone mass, increase muscle mass, increase lean body mass, decrease fat body mass, and treat bone fracture and muscle damage in mammals, including humans.

### FIELD OF THE INVENTION

This invention relates to novel amino substituted benzonitrile compounds and to pharmaceutical compositions containing the novel amino substituted benzonitrile compounds. This invention also relates to methods of treatment using amino substituted benzonitrile derivatives to prevent and/or restore age-related decline in muscle mass and strength, treat a wasting disease, treat a condition that prevents with low bone mass, increase muscle mass, increase lean body mass, decrease fat body mass, and treat bone fracture and muscle damage in mammals, including humans.

### BACKGROUND OF THE INVENTION

The worldwide population over 65 years of age is the most rapidly expanding segment of the population. A significant problem for the elderly is the decline in muscle mass and strength leading to frailty, the loss of independence, and eventual institutionalization. In the U.S. today, 1.5 million persons aged 65+ years are institutionalized and 33% of these individuals are put into long term healthcare facilities solely due to their physical frailty and their inability to maintain prerequisite activities of daily living. The frail elderly are in need of a therapy either to prevent or restore the loss of age-related muscle mass and strength. There are no therapies currently approved for the treatment of frailty. Further, the only option available to the physician is androgen replacement therapy, but its non-selective tissue action has resulted in many unacceptable side effects.

Concomitant with the age-related decline in muscle mass and strength is the loss of bone mass. Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including spine, hip and wrist fractures. Hip fractures are the most serious consequence of osteoporosis, with 5-20% of patients dying within one year, and over 50% of survivors being incapacitated.

The elderly are at greatest risk of osteoporosis, and the problem is therefore predicted to increase significantly with the aging of the population. Worldwide fracture incidence is forecasted to increase three-fold over the next 60 years, and one study estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake.

There are currently two main types of pharmaceutical therapy for the treatment of osteoporosis. The first is the use of anti-resorptive compounds to reduce the resorption of bone tissue.

A second type of pharmaceutical therapy for the treatment of osteoporosis is the use of anabolic agents to promote bone formation and increase bone mass. This class of agents is expected to restore bone to the established osteoporotic skeleton.

Intracellular receptors (IRs) form a class of structurally-related genetic regulators scientists have named “ligand dependent transcription factors.” (R. M. Evans, 240 Science, 889 1988). Steroid receptors are a recognized subset of the IRs, including the androgen receptor (AR). Regulation of a gene by such factors requires both the IR itself and a corresponding ligand which has the ability to selectively bind to the IR in a way that affects gene transcription.

Japanese Unexamined Patent Application (Kokai) No. 2002-88073 discloses cyanophenyl derivatives with antiandrogenic activity useful for the treatment or prevention of prostate cancer and prostamegaly. PCT International Patent Application WO 00/17163 discloses a series of piperazino-substituted cyanophenyl derivatives which exhibit antiandrogenic activity and are useful for the prevention or treatment of prostatic cancer and prostatic hypertrophy.

Although there are a variety of osteoporosis therapies, there is a continuing need and a continuing search in this field of art for alternative osteoporosis therapies. In addition, there is a need for bone fracture healing therapies and therapies for maintaining or increasing muscle mass, increasing lean body mass, decreasing fat body mass and promoting muscle damage repair. There also is a need for therapy that can promote bone re-growth.

### SUMMARY OF THE INVENTION

This invention is directed to methods of treatment using amino substituted benzonitrile compounds of Formula I 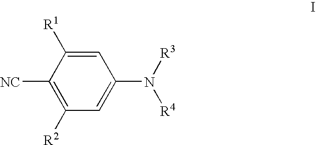
a prodrug thereof or a pharmaceutically acceptable salt of said compound or said prodrug, wherein:
R¹ is hydrogen;
R² is chloro, cyano or trifluoromethyl;
or R¹ and R² are each fluoro;
R³ and R⁴ are each independently (C₁-C₆)alkyl, (C₃-C₇)cycloalkyl or (C₂-C₆)alkenyl; or R³ and R⁴ taken together with the nitrogen to which they are attached is 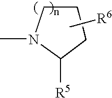
n is 1, 2 or 3;
R⁵ is (C₁-C₆)alkyl optionally substituted with hydroxy or (C₁-C₆)alkoxy; and
R⁶ is hydrogen or (C₁-C₆)alkyl optionally substituted with a hydroxy or (C₁-C₆)alkoxy.

A particular aspect of this invention is directed to methods for treating or preventing a condition that presents with low bone mass in a mammal (including a human being) which comprise administering to a mammal in need of such treatment a therapeutically effective amount of a Formula I compound or a pharmaceutically acceptable salt of said compound.

This invention is particularly directed to such methods wherein the condition that presents with low bone mass is osteoporosis, frailty, an osteoporotic fracture, a bone defect, childhood idiopathic bone loss, alveolar bone loss, mandibular bone loss, bone fracture, osteotomy, periodontitis or prosthetic ingrowth.

A particular aspect of this invention is directed to methods for treating osteoporosis in a mammal (including a human being) which comprise administering to a mammal in need of such treatment a therapeutically effective amount of a Formula I compound or a pharmaceutically acceptable salt of said compound.

Another aspect of this invention is directed to methods for treating a bone fracture or an osteoporotic fracture in a mammal which comprise administering to a mammal in need of such treatment a therapeutically effective amount of a Formula I compound or a pharmaceutically acceptable salt of said compound.

Yet another aspect of this invention is directed to methods of concomitantly treating bone fracture and muscle damage in a mammal which comprise administering to a mammal in need of such treatment a therapeutically effective amount of a Formula I compound or a pharmaceutically acceptable salt of said compound.

Yet another aspect of this invention is directed to methods for treating wasting diseases in a mammal (including a human being, either male or female) which comprise administering to a mammal in need of such treatment a therapeutically effective amount of a Formula I compound or a pharmaceutically acceptable salt of said compound.

Yet another aspect of this invention is directed to methods for the prevention and/or restoration of the age-related decline in muscle mass and strength in a mammal (including a human being) which comprise administering to a mammal in need of such treatment a therapeutically effective amount of a Formula I compound or a pharmaceutically acceptable salt of said compound.

Yet another aspect of this invention is directed to methods for increasing muscle mass in a mammal (including a human being) which comprise administering to a mammal in need of such treatment a therapeutically effective amount of a Formula I compound or a pharmaceutically acceptable salt of said compound.

Yet another aspect of this invention is directed to methods for increasing lean body mass in a mammal (including a human being) which comprise administering to a mammal in need of such treatment a therapeutically effective amount of a Formula I compound or a pharmaceutically acceptable salt of said compound.

Yet another aspect of this invention is directed to methods for decreasing fat body mass in a mammal (including a human being) which comprise administering to a mammal in need of such treatment a therapeutically effective amount of a Formula I compound or a pharmaceutically acceptable salt of said compound.

The methods of increasing muscle mass, increasing lean body mass or decreasing fat body mass may be employed for livestock animals, such as cattle, pigs and sheep, or for companion animals, such as dogs and cats. In the case of livestock animals such methods can be used to enhance growth rates and improve meat quality.

Yet another aspect of this invention is directed to methods for treating frailty in a mammal (including a human being) which comprises administering to a mammal in need of such treatment a therapeutically effective amount of a Formula I compound or a pharmaceutically acceptable salt of said compound.

Yet another aspect of this invention is directed to methods for treating acne in a mammal (including a human being) which comprise administering to a mammal in need of such treatment a therapeutically effective amount of a Formula I compound or a pharmaceutically acceptable salt of said compound.

Yet another aspect of this invention is directed to methods for treating male-pattern baldness in a human being which comprise administering to a human in need of such treatment a therapeutically effective amount of a Formula I compound or a pharmaceutically acceptable salt of said compound.

Yet another aspect of this invention is directed to methods for treating male hormone deficiency in a mammal (including a human being) which comprise administering to a mammal in need of such treatment a therapeutically effective amount of a Formula I compound or a pharmaceutically acceptable salt of said compound.

Yet another aspect of this invention is directed to methods for treating hirsutism in a mammal (including a human being) which comprise administering to a mammal in need of such treatment a therapeutically effective amount of a Formula I compound or a pharmaceutically acceptable salt of said compound.

Yet another aspect of this invention is directed to methods for treating hematopoiesis in a mammal (including a human being) which comprise administering to a mammal in need of such treatment a therapeutically effective amount of a Formula I compound or a pharmaceutically acceptable salt of said compound.

Yet another aspect of this invention is directed to methods for treating hypogonadism in a mammal (including a human being) which comprise administering to a mammal in need of such treatment a therapeutically effective amount of a Formula I compound or a pharmaceutically acceptable salt of said compound.

Yet another aspect of this invention is directed to methods for treating prostatic hyperplasia in a mammal (including a human being) which comprise administering to a mammal in need of such treatment a therapeutically effective amount of a Formula I compound or a pharmaceutically acceptable salt of said compound.

Yet another aspect of this invention is directed to methods for treating hormone dependent cancers in a mammal (including a human being) which comprise administering to a mammal in need of such treatment a therapeutically effective amount of a Formula I compound or a pharmaceutically acceptable salt of said compound.

Yet another aspect of this invention is directed to methods for treating prostate cancer in a mammal (including a human being) which comprise administering to a mammal in need of such treatment a therapeutically effective amount of a Formula I compound or a pharmaceutically acceptable salt of said compound.

Yet another aspect of this invention is directed to methods for treating breast cancer in a mammal (including a human being) which comprise administering to a mammal in need of such treatment a therapeutically effective amount of a Formula I compound or a pharmaceutically acceptable salt of said compound.

Yet another aspect of this invention is directed to methods for promoting anabolic activity in a mammal (including a human being) which comprise administering to a mammal in need of such treatment a therapeutically effective amount of a Formula I compound or a pharmaceutically acceptable salt of said compound.

Yet another aspect of this invention is directed to methods for treating obesity in a mammal (including a human being) which comprise administering to a mammal in need of such treatment a therapeutically effective amount of a Formula I compound or a pharmaceutically acceptable salt of said compound.

In the methods of this invention the compound of Formula I is administered systemically, e.g. orally, subcutaneously, intramuscularly, perenterally, transdermally or via aerosol. Alternatively, in the methods of this invention the compounds of Formula I are administered locally.

Yet another aspect of this invention is directed to certain compounds within the scope of Formula I wherein the compound is selected from:

4-(2-ethyl-piperidin-1-yl)-2-trifluoromethyl-benzonitrile;
4-(2-ethyl-piperidin-1-yl)-2,6-difluoro-benzonitrile;
2-chloro-4-(2-ethyl-piperidin-1-yl)-benzonitrile;
4-(2-ethyl-piperidin-1-yl)-phthalonitrile;
4-(sec-butyl-methyl-amino)-2-trifluoromethyl-benzonitrile;
4-(sec-butyl-ethyl-amino)-2-chloro-benzonitrile;
4-(sec-butyl-methyl-amino)-2-chloro-benzonitrile;
4-(sec-butyl-propyl-amino)-2-chloro-benzonitrile;
4-(sec-butyl-propyl-amino)-phthalonitrile;
4-(sec-butyl-ethyl-amino)-2-trifluoromethyl-benzonitrile;
4-(sec-butyl-methyl-amino)-phthalonitrile;
4-(sec-butyl-ethyl-amino)-phthalonitrile;
4-dipropylamino-2-trifluoromethyl-benzonitrile;
4-(ethyl-isopropyl-amino)-phthalonitrile;
4-dipropylamino-phthalonitrile;
4-diethylamino-2-trifluoromethyl-benzonitrile;
2-chloro-4-(ethyl-propyl-amino)-benzonitrile;
2-chloro-4-(isopropyl-methyl-amino)-benzonitrile;
4-[(1,2-dimethyl-propyl)-methyl-amino]-2-trifluoromethyl-benzonitrile;
2-chloro-4-[(1,2-dimethyl-propyl)-methyl-amino]-benzonitrile;
4-(2-methyl-pyrrolidin-1-yl)-2-trifluoromethyl-benzonitrile;
4-(cyclopentyl-methyl-amino)-2-trifluoromethyl-benzonitrile;
4-(2-propyl-piperidin-1-yl)-2-trifluoromethyl-benzonitrile;
4-(2-ethyl-4-hydroxymethyl-piperidin-1-yl)-2-trifluoromethyl-benzonitrile;
4-(sec-butyl-propyl-amino)-2-trifluoromethyl-benzonitrile;
4-(allyl-methyl-amino)-2-trifluoromethyl-benzonitrile;
4-(2-methyl-piperidin-1-yl)-2-trifluoromethyl-benzonitrile;
4-(2-(ethoxy-methyl)-pyrrolidin-1-yl)-2-trifluoromethyl-benzonitrile;
4-(2-(2-hydroxyethyl)-piperidin-1-yl)-2-trifluoromethyl-benzonitrile;
4-(2-(methoxy-methyl)-pyrrolidin-1-yl)-2-trifluoromethyl-benzonitrile;
4-(ethyl-pentyl-amino)-2-trifluoromethyl-benzonitrile;
4-( butyl-propyl-amino)-2-trifluoromethyl-benzonitrile;
4-(isopropyl-methyl-amino)-2-trifluoromethyl-benzonitrile;
4-(pentyl-propyl-amino)-2-trifluoromethyl-benzonitrile;
4-(2-hydroxymethyl-piperidin-1-yl)-2-trifluoromethyl-benzonitrile;
4-(butyl-ethyl-amino)-2-trifluoromethyl-benzonitrile;
4-(dibutyl-amino)-2-trifluoromethyl-benzonitrile;
4-(2-(hydroxy-methyl)-pyrrolidin-1-yl)-2-trifluoromethyl-benzonitrile;
2-chloro-4-(isopropyl-propyl-amino)-benzonitrile;
2-chloro-4-(diethyl-amino)-benzonitrile;
2-chloro-4-(ethyl-isopropyl-amino)-benzonitrile;
2-chloro-4-(dipropyl-amino)-benzonitrile;
4-(isopropyl-methyl-amino)-phthalonitrile;
4-(2-methoxymethyl-pyrrolidin-1-yl)-phthalonitrile;
4-azepan-1-yl-phthalonitrile; or 2,6-difluoro-4-(dipropyl-amino)-benzonitrile; or
   a stereoisomer thereof or a pharmaceutically acceptable salt of said compound or stereoisomer.

Yet another aspect of this invention is directed to pharmaceutical compositions comprising certain compounds within the scope of Formula I as recited directly hereinabove together with a pharmaceutically acceptable vehicle, diluent or carrier. The pharmaceutical compositions may be employed in the methods of treatment described hereinabove.

In the methods of the present invention it is preferred that the mammal is human, livestock or a companion animal.

A preferred dosage is about 0.001 to 100 mg/kg/day of a Formula I compound or a pharmaceutically acceptable salt of said compound.

An especially preferred dosage is about 0.01 to 10 mg/kg/day of a Formula I compound or a pharmaceutically acceptable salt of said compound.

The compounds of Formula I are androgen receptor modulators which have an affinity for the androgen receptor and cause a biological effect by binding to the receptor. Typically, the compounds of Formula I act as androgen receptor agonists, which may exhibit tissue selective androgen receptor agonist activity. The compounds of Formula I that exhibit androgen receptor agonist activity can be employed to treat conditions responsive to agonism of the androgen receptor.

### DETAILED DESCRIPTION OF THE INVENTION

The term “treating”, “treat” or “treatment” as used herein includes preventative (e.g., prophylactic) and palliative treatment.

By “pharmaceutically acceptable” is meant that the vehicle, carrier, diluent, excipients, and/or salt must be compatible with the other ingredients of the formulation, and not deleterious to the recipient thereof.

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

As used herein, the expressions “reaction-inert solvent” and “inert solvent” refers to a solvent or a mixture of solvents which does not interact with starting materials, reagents, intermediates or products in a manner which adversely affects the yield of the desired product.

The parenthetical negative or positive sign used herein (e.g., when found in parentheses in the name of a compound) denotes the direction a plane of polarized light is rotated by the particular stereoisomer.

The term “(C₁-C₆)alkyl” means a straight or branched alkyl group having from one to six carbons. Examples of “(C₁-C₆)alkyl” include, but are not limited to, methyl, ethyl, propyl, butyl, pentyl, hexyl, isopropyl, isobutyl, sec-butyl and neopentyl. The term “(C₃-C₇)cycloalkyl” means a cycloalkyl group having from three to seven carbons. Examples of “(C₃-C₇)cycloalkyl” include cyclopropyl, cyclobutyl, cyclopentyl, cyclohexyl and cycloheptyl. The term “(C₂-C₆)alkenyl” means a straight or branched alkenyl group having from two to six carbons. Examples of “(C₂-C₆)alkenyl” include, but are not limited to, vinyl, allyl, propenyl, butenyl, pentenyl, hexenyl, isopropenyl, isobutenyl, sec-butenyl and neopentenyl. The term “(C₁-C₆)alkoxy” means a straight or branched alkoxy group having from one to six carbons. Examples of “(C₁-C₆)alkoxy” include, but are not limited to, methoxy, ethoxy, propoxy, butoxy, pentoxy, hexoxy, isopropoxy, isobutoxy, sec-butoxy and neopentoxy.

The chemist of ordinary skill will recognize that the compounds of this invention may contain one or more atoms which may be in a particular stereochemical or geometric configuration, giving rise to stereoisomers and configurational isomers. All such isomers and mixtures thereof are included in this invention. Hydrates and solvates of the compound of this invention are also included.

It will be recognized that the compounds of this invention can exist in isotopically labelled form, i.e., said compounds may contain one or more atoms containing an atomic mass or mass number different from the atomic mass or mass number usually found in nature. Isotopes of hydrogen, carbon, phosphorous, fluorine and chlorine include H, ²H, ³H, ¹²C, ¹³C, ¹⁴C, ³¹P, ³²P, ³²S, ³⁵S, ¹⁸F, ¹⁹F, ³⁵Cl and ³⁶Cl, respectively. Compounds of this invention, a prodrug thereof, or a pharmaceutically acceptable salt of said compound or of said prodrug which contain those isotopes and/or other isotopes of other atoms are within the scope of this invention. Tritiated, i.e., ³H, and carbon-14, i.e., ¹⁴C, isotopes are particularly preferred for their ease of preparation and detectability. Further, certain deuterated, i.e., ²H, compounds may afford advantages with respect to metabolic stability and, hence, may be preferred. Isotopically labelled compounds of Formula I of this invention and prodrugs thereof can generally be prepared by carrying out the procedures disclosed in the Schemes and/or in the Examples and Preparations below by substituting a readily available isotopically labelled reagent for a non-isotopically labelled reagent.

Other features and advantages of this invention will be apparent from this specification and the appendant claims which describe the invention.

Certain processes for the manufacture of the compounds of this invention are provided as further features of the invention and are illustrated by the following description and by the examples.

Synthesis

The compounds of Formula I can be prepared by methods analogously known in the art. One method for preparing these compounds is described below in Reaction Scheme I. Reaction Scheme I describes the synthesis of a compound of Formula I by a nucleophilic substitution reaction of the tertiary amine HNR³R⁴ of formula III with an intermediate of formula II. The group LG in the intermediate of formula II represents any appropriate leaving group and typically a fluoro group is employed. The reaction can be run neat or in an appropriate reaction-inert solvent. The reaction may be run at ambient temperature or with heating. The reaction is typically carried out neat between 65° C. and 105° C. using 1.5 to 2.5 equivalents of the amine HNR³R⁴ for a period of 12 to 24 hours. Certain of the 4-fluoro-benzonitrile derivatives, of formula II wherein LG is fluoro, are known in the art and may be synthesized as described by Japanese Patent Application Number 01097937. The resulting product, a compound of Formula I, can be recovered by extraction, evaporation, or other techniques known in the art. It may then optionally be purified by chromatography, recrystallization, distillation, or other techniques known in the art. In certain cases the crude reaction mixture can be further reacted with another amine, such as 1,2-ethane-diamine, in order to consume any remaining starting material and facilitate the purification of the compound of Formula I.

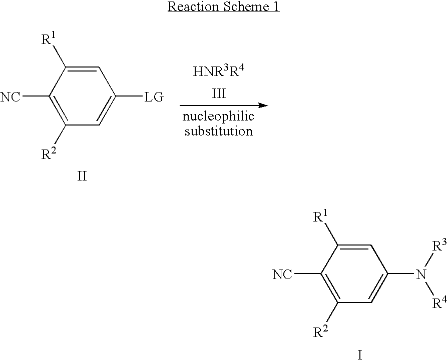

Reaction Scheme 2 describes the synthesis of a compound of Formula I by a nucleophilic substitution reaction of the secondary amine H₂NR³ of Formula IV with an intermediate of formula II to provide the intermediate of Formula V. The nucleophilic substitution reaction employing the amine H₂NR³ and the intermediate of Formula II can be carried out under the nucleophilic substitution conditions described above for Scheme 1. The resulting product, an intermediate of Formula V, can then be alkylated with an appropriate alkylating agent of formula R₄X to provide the product of Formula I. The group X in the alkylating agent R₄X represents an appropriate leaving group, such as a halide and typically an iodide. The alkylation reaction can be carried out in the presence of an appropriate base, such as sodium hydride or potassium hydride, in an appropriate aprotic solvent such as tetrahydrofuran (THF). The alkylation reaction is typically carried out at ambient temperature for a period of one to twenty four hours by treating the intermediate of Formula V with two to three equivalents of an appropriate base in an appropriate solvent followed by addition of two equivalents of the alkylating agent R₄X. The reaction mixture can be quenched by addition of water and the product of Formula I can be recovered by extraction, evaporation, or other techniques known in the art. It may then optionally be purified by chromatography, recrystallization, distillation, or other techniques known in the art.

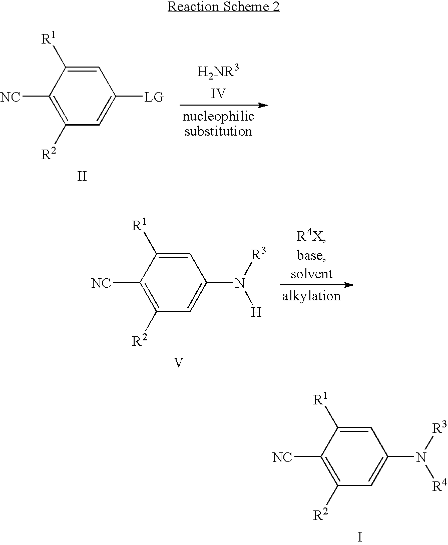

As would be appreciated by those skilled in the art, some of the methods useful for the preparation of such compounds, as discussed above, may require protection of a particular functionality, e.g., to prevent interference by such functionality in reactions at other sites within the molecule or to preserve the integrity of such functionality. The need for, and type of, such protection is readily determined by one skilled in the art, and will vary depending on, for example, the nature of the functionality and the conditions of the selected preparation method. See, e.g., T. W. Greene, Protective Groups in Organic Synthesis, John Wiley & Sons, New York, 1991.

Some of the compounds of this invention are acidic and they form a salt with a pharmaceutically acceptable cation. Some of the compounds of this invention are basic and they form a salt with a pharmaceutically acceptable anion. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures.

The compounds of Formula I are androgen receptor modulators which have an affinity for the androgen receptor and cause a biological effect by binding to the receptor. Typically, the compounds of Formula I act as agonists, which may exhibit tissue selective androgen receptor agonist activity. The compounds of Formula I that exhibit androgen receptor agonist activity can be employed to treat conditions responsive to agonism of the androgen receptor. Examples of such conditions include, but are not limited to, conditions that present with low bone mass, such as osteoporosis, frailty, an osteoporotic fracture, a bone defect, childhood idiopathic bone loss, alveolar bone loss, mandibular bone loss, bone fracture, osteotomy, periodontitis or prosthetic ingrowth. The term osteoporosis includes primary osteoporosis, such as senile, postmenopausal and juvenile osteoporosis, as well as secondary osteoporosis, such as osteoporosis due to hyperthyroidism or Cushing syndrome (due to corticosteroid use), acromegaly, hypogonadism, dysosteogenesis and hypophospatasemia. The compounds of the invention with androgen receptor agonist activity may also be employed for treating wasting diseases (such as post operative, tumor, trauma, chronic renal disease or AIDS induced), male hypogonadism, male sexual dysfunction (male erectile dysfunction, male dysspermatogenic sterility), abnormal sex differentiation (male hermaphroditism), male delayed puberty, male infertility, aplastic anemia, hemolytic anemia, sickle cell anemia, renal anemia, idiopathic thrombocytopenic purpura, myelofibrosis, inoperable breast cancer or mastopathy. The compounds of the invention with androgen receptor agonist activity may also be used to increase muscle mass, increase lean body mass, decrease fat body mass or treat concomitant bone fracture and muscle damage. The use of compounds of the invention with androgen receptor agonist activity may also be used to increase muscle mass, increase lean body mass and decrease fat body mass in non-mammals such as birds and fish. A preferred method to increase muscle mass, increase lean body mass and decrease fat body mass in a non-mammal is the use of a compound of formula I in poultry, such as chickens and turkeys.

Certain of the compounds of Formula I may exhibit androgen receptor antagonist activity and may further exhibit tissue selective androgen receptor antagonist activity. Compounds acting as androgen receptor antagonists may be used to treat hormone dependent cancers such as prostate carcinomas, benign prostatic hyperplasia, acne, hirsutism, excess sebum production, alopecia, hypertrichosis, precocious puberty, prostamegaly, virilization and polycystic ovary syndrome.

Compounds of formula I acting as androgen receptor antagonists may also be used to improve meat quality in livestock animals. Intact male livestock animals can exhibit growth advantages associated with male sex steroids such as increased feed conversion, increased growth rate, and increased carcass quality (leanness) when compared with castrated male livestock animals. In addition, the intact male livestock animals can exhibit decreased nitrogenous waste output compared with castrated livestock animals and intact males are not at risk of infection associated with castration. Intact male livestock animals can, however, exhibit increased levels of taint when compared to castrated male livestock animals due to higher levels of testosterone, androstenone, skatole and indole in the intact animals. The compounds of formula I with androgen receptor antagonist activity can be administered to male livestock animals during and/or at the end of the growing period to allow for the growth advantages associated with male sex steroids during the growth period while eliminating taint of the livestock meat upon slaughter. A preferred method of improving meat quality in a male livestock animal is the administration of a compound of formula I to a boar at the end of the growing period in order to eliminate boar taint.

Administration of a compound of this invention can be via any method that delivers the compound systemically and/or locally. These methods include, but are not limited to, oral routes, parenteral, transdermal and intraduodenal routes, etc. Generally, a compound of this invention is administered orally, but parenteral administration (e.g., intravenous, intramuscular, subcutaneous or intramedullary) may be utilized, for example, where oral administration is inappropriate for the target or where the patient is unable to ingest the drug.

In general an effective dosage for the Formula I compound of this invention and the salts of the compound is in the range of 0.001 to 100 mg/kg/day, preferably 0.01 to 10 mg/kg/day.

Furthermore, it will be understood by those skilled in the art that a compound of the present invention, including pharmaceutical compositions and formulations containing the compound or a salt thereof, can be used in a wide variety of combination therapies to treat the conditions and diseases described above. Thus, a compound or a salt thereof of the present invention can be used in combination with other hormones and other therapies, including, without limitation, chemotherapeutic agents such as cytostatic and cytotoxic agents, immunological modifiers such as interferons, interleukins, growth hormones and other cytokines, hormone therapies, surgery and radiation therapy.

A compound or a salt thereof of the present invention is generally administered in the form of a pharmaceutical composition comprising the compound or a salt thereof together with a pharmaceutically acceptable vehicle, diluent or carrier. Thus, the compound of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example olive oil, Miglyol® or Capmul®, in a soft gelatin capsule. Antioxidants may be added to prevent long term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of compound or a salt thereof of this invention, preferably 1%-70%. In any event, the composition or formulation to be administered will contain a quantity of a compound or a salt thereof according to the invention in an amount effective to treat the disease/condition of the subject being treated.

All documents cited in this application, including patents and patent applications, are hereby incorporated by reference. The examples presented below are intended to illustrate particular embodiments of the invention and are not intended to limit the invention, including the claims, in any manner.

### EXAMPLES

The following abbreviations, when used in this application, have the following meanings.
NMR nuclear magnetic resonance
H hydrogen
s singlet
d doublet
t triplet
m multiplet
bm broad multiplet
MS mass spectra
LCMS liquid chromatography mass spectrometry
APCI⁺ atmospheric pressure chemical ionization (positive mode)
HPLC high pressure liquid chromatography
SEM standard error measurement

The compound or a salt thereof of this invention either alone or in combination with other compounds as described hereinabove generally will be administered in a convenient formulation. The following formulation examples only are illustrative and are not intended to limit the scope of the present invention.

In the formulations that follow, “active ingredient” means a compound or a salt thereof of this invention.

Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet formulation is prepared using the ingredients below:

Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredient, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol

| Ingredient | Quantity (% by weight) |
|---|---|
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| Ingredient | Quantity (mg/suppository) |
|---|---|
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | ml |

The solution of the above ingredients is intravenously administered to a patient at a rate of about 1 mL per minute.

Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyol ® Oil | 500-1000 |

The active ingredient above may also be a combination of agents.

### Preparation 1

### Preparation of (+)-2-ethyl-piperidine by resolution of 2-ethyl-piperidine

(R)-(1)-mandelic acid (40 g, 265 mmol.) and 2-ethyl-piperidine (30 g, 265 mmol.) were dissolved in methanol (100 mL). The mixture was warmed gently to ensure all material was in solution and then it was cooled to 0° C. Diethyl ether (230 mL) was added slowly to the cooled solution and it was allowed to sit for 24 hours at 0° C. The resulting white crystals were isolated and dried under high vacuum. The resulting salt was dissolved in warm methanol. Diethyl ether was added and the resulting solution was cooled to 0° C. to afford the desired crystalline product. The isolated mandelic acid salt of (+)-2-ethyl-piperidine was dissolved in cooled H₂O and solid potassium hydroxide was added to bring the pH of the solution to 14. The (+)-2-ethyl-piperidine (15.88 g) was extracted with diethyl ether (3×), dried (MgSO₄), filtered, and concentrated to a clear oil. [α]₅₈₉+4.88° (0.413 g/mL, CHCl₃). ¹H NMR (CDCl₃)δ: 0.7 (m, 3H), 0.9 (m, 1H), 1.2 (bm, 3H), 1.3 (bm, 1H), 1.4 (bm, 1H), 1.6 (m, 1H), 2.0 (bm, 1H), 2.2 (bm, 1H), 2.4 (bm, 1H), 2.9 (bm, 1H).

### Example 1

### 4-(2-(S)-Ethyl-piperidin-1-yl)-2-trifluoromethyl-benzonitrile

(+)-2-Ethyl-piperidine (3.0 g, 26.5 mmol., Preparation 1) and 4-fluoro-2-trifluoromethyl-benzonitrile (2.0 g, 10.6 mmol.) were heated neat at 65° C. overnight. The reaction mixture was cooled and partitioned between diethyl ether and 1N HCl. The organic layers were combined, dried (MgSO₄), filtered, and evaporated to dryness. The resulting residue (0.5 g, 2.64 mmol.) was a mixture of desired product and starting 4-fluoro-2-trifluoromethyl-benzonitrile. This mixture was treated with ethane-1,2-diamine (0.64 g, 10.58 mmol.) and heated at 80° C. for 3 days. The reaction mixture was cooled and partitioned between diethyl ether and 0.5 N HCl. The organic layer was washed with 0.5N HCl (5×), dried (MgSO₄), filtered, and evaporated to dryness. The resulting yellow oil was purified via Biotage™ Flash 40 (Biotage Inc., Charlottesville, Va., USA) chromatography using 10% ethyl acetate/hexanes as the eluant to afford the desired title product (11.8 g) as a yellow oil. MS (LCMS⁺) Calc.: 282.3, Found: 283.4 (M+1). ¹H NMR (CDCl₃)δ: 0.9 (t, 3H), 1.6 (m, 8H), 3.1 (m, 1H), 3.6 (bm, 1H), 3.9 (bm, 1H), 6.9 (bm, 1H), 7.0 (s, 1H), 7.5 (m, 1H).

### Example 2

### 4-(2-(S)-Ethyl-piperidin-1-yl)-2,6-difluoro-benzonitrile

A procedure analogous to the procedure described in Example 1 was followed by reacting 2,4,6-trifluoro-benzonitrile with (+)-2-ethyl-piperidine at 105° C. to afford the crude product. The crude residue was purified by Chromatotron® centrifugal thin-layer chromatograph (2000μ, Harrison Research, Inc., Palo Alto, Calif., USA) using 2% ethyl acetate/hexanes as the eluant to yield the title compound. MS (APCI⁺) Calc.: 250.2, Found: 251.2 (M+1). ¹H NMR (CDCl₃)δ: 0.9 (m, 3H), 1.7 (bm, 8H), 3.0 (m, 1H), 3.5 (m, 1H), 3.8 (m, 1H), 6.3 (m, 2H).

### Example 3

### 2-Chloro-4-(2-(S)-ethyl-piperidin-1-yl)-benzonitrile

A procedure analogous to the procedure described in Example 1 was followed by reacting 2-chloro-4-fluoro-benzonitrile with (+)-2-ethyl-piperidine at 105° C. to afford the crude product. The crude residue was purified by Chromatotron® (2000μ) using 2% ethyl acetate/hexanes as the eluant to yield the title compound. MS (APCI⁺) Calc.: 248.8, Found: 249.2 (M+1). ¹H NMR (CDCl₃)δ: 0.9 (m, 3H), 1.7 (bm, 8H), 3.0 (m, 1H), 3.6 (m, 1H), 3.8 (m, 1H), 6.7 (m, 1H), 6.8 (m, 1H), 7.4 (m, 1H).

### Example 4

### 4-(2-(S)-Ethyl-piperidin-1-yl)-phthalonitrile

A procedure analogous to the procedure described in Example 1 was followed by reacting 4-fluoro-phthalonitrile with (+)-2-ethyl-piperidine at 105° C. to afford the crude product. The crude residue was purified by Chromatotron® (2000μ) using 2% ethyl acetate/hexanes as the eluant to yield the title compound. MS (APCI⁺) Calc.: 239.3, Found: 240.2 (M+1). ¹H NMR (CDCl₃)δ: 0.9 (m, 3H), 1.7 (bm, 8H), 3.0 (m, 1H), 3.6 (m, 1H), 3.9 (m, 1H), 6.9 (m, 1H), 7.0 (m, 1H), 7.5 (m, 1H).

### Example 5

### 4-(2-Ethyl-piperidin-1-yl)-phthalonitrile

A procedure analogous to the procedure described in Example 1 was followed by reacting 4-fluoro-phthalonitrile with 2-ethyl-piperidine at 75° C. to afford the crude product. The crude residue was purified by Chromatotron® (2000 μ) using 2% ethyl acetate/hexanes as the eluant to yield the title compound. MS (APCI⁺) Calc.: 239.3, Found: 240.3 (M+1). ¹H NMR (CDCl₃)δ: 0.9 (m, 3H), 1.7 (bm, 8H), 3.1 (m, 1H), 3.6 (m, 1H), 3.9 (m, 1H), 6.9 (m, 1H), 7.1 (m, 1H), 7.5 (m, 1H).

### Example 6

### 4-(2-Ethyl-piperidin-1-yl)-2-trifluoromethyl-benzonitrile

The general procedure described in Example 1 was followed by reacting 4-fluoro-2-trifluoromethyl-benzonitrile with 2-ethyl-piperidine at 70° C. to yield the desired crude product. The reaction mixture was cooled and partitioned between dichloromethane and 2M HCl. The organic layer was dried (MgSO₄), filtered, and concentrated to dryness. The residue was purified by preparative thin-layer chromatography (TLC) using 30% ethyl acetate/hexanes as the eluant to give the title compound. MS (APCI⁺) Calc.: 282.3, Found: 283.2 (M+1). ¹H NMR (CDCl₃)δ: 0.9 (m, 3H), 1.7 (bm, 8H), 3.0 (m, 1H), 3.7 (m, 1H), 3.9 (m, 1H), 6.9 (m, 1H), 7.1 (m, 1H), 7.6 (m, 1H).

### Example 7

### 4-((R)-sec-Butyl-methyl-amino)-2-trifluoromethyl-benzonitrile

Step A: Preparation of 4-(R)-sec-Butylamino-2-trifluoromethyl-benzonitrile

Ethyl-diisopropyl-amine (7.1 g, 109.38 mmol.), (−)-sec-butylamine (6.0 g, 82.03 mmol.), and 4-fluoro-2-trifluoromethyl-benzonitrile (10.3 g, 54.69 mmol.) were combined and heated overnight at 60° C. The reaction mixture was cooled and partitioned between diethyl ether and 1N HCl. The organic layer was dried (MgSO₄), filtered, and concentrated to dryness. The resulting yellow liquid formed white crystals upon standing. The crystalline material was isolated by filtration and found to be pure desired product. The mother liquor was heated with hexanes and allowed to cool to ambient temperature for recrystallization. A total of 7.39 g of white crystalline product was obtained. ¹H NMR (CDCl₃)δ: 1.0 (t, 3H), 1.2 (d, 3H), 1.6 (m, 2H), 3.5 (m, 1H), 4.4 (bm, 1H), 6.6 (m, 1H), 6.8 (s, 1H), 7.5 (m, 1H).

Step B: Preparation of 4-((R)-sec-Butyl-methyl-amino)-2-trifluoromethyl-benzonitrile

4-(R)-sec-butylamino-2-trifluoromethyl-benzonitrile (2.6 g, 10.73 mmol.) dissolved in tetrahydrofuran (THF) (60 mL) and the resulting solution was added to a flask containing a 60% dispersion of sodium hydride in mineral oil (0.515 9, 21.47 mmol.) and the mixture was stirred for 10 minutes at ambient temperature. Iodomethane (3.05 g, 21.47 mmol.) was then added to the reaction mixture and stirring was continued at ambient temperature overnight. The reaction mixture was cooled to 0° C., quenched with H₂O, and extracted with diethyl ether. The organic layers were combined, washed with brine, dried (MgSO₄), filtered, and concentrated to dryness. The crude material was purified via Biotage™ Flash 40 chromatography using 5% -10% ethyl acetate/hexanes as the gradient eluant to obtain the title compound (2.5 g) as a light yellow oil. MS (LCMS⁺) Calc.: 256.3, Found: 257.2 (M+1). ¹H NMR (CDCl₃)δ: 0.9 (t, 3H), 1.2 (m, 3H), 1.6 (m, 2H), 2.8 (s, 3H), 3.9 (m, 1H), 6.8 (m, 1H), 7.0 (m, 1H), 7.5 (m, 1H).

### Example 8

### 4-((R)-sec-Butyl-ethyl-amino)-2-chloro-benzonitrile

Step A: Preparation of 4-(R)-sec-Butylamino-2-chloro-benzonitrile

Following the general procedure in Example 7, Step A, 2-chloro-4-fluoro-benzonitrile was reacted with R-(−)-sec-butylamine to give the desired crude product. The crude material was purified by silica gel chromatography using 2% ethyl acetate/hexanes as the eluant to yield the desired pure product.

Step B: Preparation of 4-((R)-sec-Butyl-ethyl-amino)-2-chloro-benzonitrile

A 35% dispersion of potassium hydride in mineral oil (514 mg, 4.5 mmol.) was washed with hexanes (2×) under an inert atmosphere. 4-(R)-sec-butylamino-2-chloro-benzonitrile (308 mg, 1.5 mmol.) was dissolved in THF (5 mL) and added to the flask containing the potassium hydride under an inert atmosphere. The reaction mixture was stirred at ambient temperature and treated with iodoethane (0.24 mL, 3.0 mmol.). The reaction mixture was quenched slowly with H₂O and extracted with diethyl ether. The organic layer was dried (MgSO₄), filtered, and evaporated to dryness. The crude product was purified by silica gel chromatography using 2% ethyl acetate/hexanes as the eluant to afford the desired compound. This material was further purified by Shimadzu® preparative HPLC (Shimadzu Scientific Instruments, Columbia, Md., USA, a subsidiary of Shimadzu Corporation, Kyoto, Japan) using a Waters Symmetry® C8, 5 μm, 19 mm×50 mm column (Waters Corporation, Milford, Mass., USA). The gradient eluant used was 15% to 0% acetonitrile, 0.1% formic acid in water to give the title compound. MS (APCI⁺) Calc.: 236.8, Found: 237.2 (M+1). ¹H NMR (CDCl₃)δ: 0.8 (m, 3H), 1.2 (m, 6H), 1.6 (m, 1H), 1.7 (m, 1H), 3.3 (m, 2H), 3.8 (m, 1H), 6.7 (m, 1H), 6.8 (m, 1H), 7.4 (m, 1H).

The compounds of Examples 9, 10, 12, 13, 14 and 27 were prepared by following the general procedure of Example 8, Step B, but substituting appropriate benzonitrile derivative and alkyl iodide. The compounds of Examples 15, 16, 17, 18, 19, 20 and 22-23 were prepared by following the general procedure of Example 8, Step A, but substituting the appropriate benzonitrile derivative and appropriate amine. Examples 11, 21 and 24-26 were prepared by following the general procedure of Example 8, Steps A and B, but substituting the appropriate benzonitrile derivative, amine and alkyl iodide.

### Example 9

### 4-((R)-sec-Butyl-methyl-amino)-2-chloro-benzonitrile

4-(R)-sec-butylamino-2-chloro-benzonitrile was reacted with iodomethane to yield the title compound. MS (APCI⁺) Calc.: 222.7, Found: 223.2 (M+1). ¹H NMR (CDCl₃)δ: 0.8 (t, 3H), 1.1 (m, 3H), 1.6 (m, 2H), 2.7 (s, 3H), 3.8 (m, 1H), 6.6 (m, 1H), 6.7 (m, 1H), 7.4 (m, 1H).

### Example 10

### 4-((R)-sec-Butyl-propyl-amino)-2-chloro-benzonitrile

4-(R)-sec-butylamino-2-chloro-benzonitrile was reacted with iodopropane to afford the title compound. MS (APCI⁺) Calc.: 250.8, Found: 251.2 (M+1). ¹H NMR (CDCl₃)δ: 0.8 (m, 6H), 1.2 (m, 3H), 1.6 (m, 4H), 3.1 (m, 2H), 3.8 (m, 1H), 6.5 (m, 1H), 6.7 (s, 1H), 7.4 (m, 1H).

### Example 11

### 4-((R)-sec-Butyl-propyl-amino)-phthalonitrile

Step A: Preparation of 4-(R)-sec-Butylamino-phthalonitrile

4-Fluoro-phthalonitrile was reacted with (−)-sec-butylamine to afford the desired compound.

Step B: Preparation of 4-((R)-sec-Butyl-propyl-amino)-phthalonitrile

4-(R)-sec-butylamino-phthalonitrile was reacted with iodopropane to afford the title compound. MS (APCI⁺) Calc.: 241.3, Found: 242.2 (M+1). ¹H NMR (CDCl₃)δ: 0.8 (t, 3H), 0.9 (t, 3H), 1.2 (d, 3H), 1.6 (bm, 4H), 3.1 (m, 2H), 3.8 (m, 1H), 6.8 (m, 1H), 6.9 (s, 1H), 7.5 (m, 1H).

### Example 12

### 4-((R)-sec-Butyl-ethyl-amino)-2-trifluoromethyl-benzonitrile

4-(R)-sec-butylamino-2-trifluoromethyl-benzonitrile was reacted with iodoethane to afford the title compound. MS (APCI⁺) Calc.: 270.3, Found: 271.3 (M+1). ¹H NMR (CDCl₃)δ: 0.9 (t, 3H), 1.2 (m, 6H), 1.6 (m, 2H), 3.3 (m, 2H), 3.9 (m, 1H), 6.8 (m, 1H), 6.9 (s, 1H), 7.5 (m, 1H).

### Example 13

### 4-((R)-sec-Butyl-methyl-amino)-phthalonitrile

4-(R)-sec-butylamino-phthalonitrile was reacted with iodomethane to afford the title compound. MS (APCI⁺) Calc.: 213.2, Found: 214.2 (M+1). ¹H NMR (CDCl₃)δ: 0.82 (t, 3H), 1.20 (t, 3H), 1.60 (m, 1H), 3.83 (m, 1H), 6.88 (dd, 1H), 6.98 (d, 1H), 7.52 (d, 1H).

### Example 14

### 4-(R)-sec-Butyl-ethyl-amino)-phthalonitrile

4-(R)-sec-butylamino-phthalonitrile was reacted with iodoethane to afford the title compound. MS (APCI⁺) Calc.: 227.3, Found 228.2 (M+1). ¹H NMR (CDCl₃)δ: 0.9 (t, 3H), 1.2 (bm, 6H), 1.6 (bm, 2H), 3.3 (m, 2H), 3.8 (m, 1H), 6.9 (m, 1H), 7.0 (s, 1H), 7.5 (m, 1H).

### Example 15

### 4-Dipropylamino-2-trifluoromethyl-benzonitrile

4-Fluoro-2-trifluoromethyl-benzonitrile was reacted with dipropylamine at 65° C. to afford the title compound. MS (APCI⁺) Calc.: 270.3, Found: 271.2 (M+1). ¹H NMR (CDCl₃)δ: 0.9 (m, 6H), 1.6 (m, 4H), 3.3 (m, 4H), 6.7 (m, 1H), 6.8 (m, 1H), 7.5 (m, 1H).

### Example 16

### 4-(Ethyl-isopropyl-amino)-phthalonitrile

4-Fluoro-phthalonitrile was reacted with ethyl-isopropyl-amine at 75° C. to afford the title compound. MS (APCI⁺) Calc.: 213.3, Found: 214.2 (M+1). ¹H NMR (CDCl₃)δ: 0.87 (t, 3H), 1.6 (m, 6H), 3.1 (m, 1H), 3.6 (m, 1H), 3.92 (m, 1H), 6.98 (m, 1H), 7.02 (m, 1H), 7.52 (m, 1H).

### Example 17

### 4-(sec-Butyl-methyl-amino)-2-trifluoromethyl-benzonitrile

4-Fluoro-2-trifluoromethyl-benzonitrile was reacted with sec-butyl-methyl-amine at 70° C. to give the desired product. The crude product was purified via Chromatotron® (2000μ) using 30% ethyl acetate/hexanes as the eluant. MS (APCI⁺) Calc.: 256.3, Found: 257.2 (M+1). ¹H NMR (CDCl₃)δ: 0.9 (m, 3H), 1.2 (m, 3H), 1.6 (m, 2H), 2.8 (s, 1H), 3.9 (m, 1H), 6.8 (m, 1H), 7.0 (m, 1H), 7.6 (m, 1H).

### Example 18

### 4-(sec-Butyl-propyl-amino)-phthalonitrile

4-Fluoro-phthalonitrile was heated with sec-butyl-propyl-amine at 105° C. to afford the title compound. MS (APCI⁺) Calc.: 241.3, Found: 242.3 (M+1). ¹H NMR (CDCl₃)δ: 0.9 (m, 3H), 1.0 (m, 3H), 1.2 (m, 3H), 1.6 (m, 4H), 3.1 (m, 2H), 3.8 (m, 1H), 6.8 (m, 1H), 6.9 (m, 1H), 7.5 (m, 1H).

### Example 19

### 4-Dipropylamino-phthalonitrile

4-Fluoro-phthalonitrile was heated with di-n-propyl amine at 105° C. to afford the title compound. MS (APCI⁺) Calc.: 227.3, Found: 228.2 (M+1). ¹H NMR (CDCl₃)δ: 0.9 (m, 6H), 1.6 (m, 4H), 3.3 (m, 4H), 6.7 (m, 1H), 6.8 (m, 1H), 7.5 (m, 1H).

### Example 20

### 4-Diethylamino-2-trifluoromethyl-benzonitrile

4-Fluoro-2-trifluoromethyl-benzonitrile was reacted with diethyl amine at 65° C. to afford the title compound. MS (APCI⁺) Calc.: 242.2, Found: 243.2 (M+1). ¹H NMR (CDCl₃)δ: 1.2 (m, 6H), 3.4 (m, 4H), 6.7 (m, 1H), 6.9 (m, 1H), 7.5 (m, 1H).

### Example 21

### 4-(sec-Butyl-ethyl-amino)-2-trifluoromethyl-benzonitrile

Step A: Preparation of 4-sec-butylamino-2-trifluoromethyl-benzonitrile

Fluoro-2-trifluoromethyl-benzonitrile was reacted with sec-butylamine to afford 4-sec-butylamino-2-trifluoromethyl-benzonitrile.

Step B: Preparation of 4-(sec-butyl-ethyl-amino)-2-trifluoromethyl-benzonitrile

4-sec-butylamino-2-trifluoromethyl-benzonitrile was reacted with iodoethane to yield the crude compound. The crude material was purified by Chromatotron® (4000μ) using 15% ethyl acetate/hexanes as the eluant to afford the title compound. MS (APCI⁺) Calc.: 270.3, Found: 271.4 (M+1). ¹H NMR (CDCl₃)δ: 0.9 (t, 3H), 1.2 (m, 6H), 1.6 (m, 2H), 3.3 (m, 2H), 3.9 (m, 1H), 6.8 (m, 1H), 7.0 (s, 1H), 7.5 (m, 1H).

### Example 22

### 2-Chloro-4-(ethyl-propyl-amino)-benzonitrile

2-Chloro-4-fluoro-benzonitrile was reacted with ethyl-propyl-amine at 90° C. to afford the title compound. MS (APCI⁺) Calc.: 222.7, Found: 223.2 (M+1). ¹H NMR (CDCl₃)δ: 0.9 (t, 3H), 1.1 (t, 3H), 1.6 (m, 2H), 3.2 (m, 2H), 3.4 (m, 2H), 6.5 (m, 1H), 6.6 (s, 1H), 7.4 (m, 1H).

### Example 23

### 2-Chloro-4-(isopropyl-methyl-amino)-benzonitrile

2-Chloro-4-fluoro-benzonitrile was reacted with isopropyl-methyl-amine at 105° C. to afford the title compound. MS (APCI⁺) Calc.: 208.7, Found: 209.1 (M+1). ¹H NMR (CDCl₃)δ: 1.2 (m, 6H), 2.8 (s, 3H), 4.1 (m, 1H), 6.6 (m, 1H), 7.4 (m, 1H).

### Example 24

### 4-[(1-(R), 2-Dimethyl-propyl)-methyl-amino]-2-trifluoromethyl-benzonitrile

Step A: Preparation of 4-(1-(R), 2-dimethyl-propylamino)-2-trifluoromethyl-benzonitrile

4-Fluoro-2-trifluoromethyl-benzonitrile was reacted with 1-(R), 2-dimethyl-propylamine at 90° C. to afford the desired product.

Step B: Preparation of 4-[(1-(R), 2-dimethyl-propyl)-methyl-amino]-2-trifluoromethyl-benzonitrile

4-(1,2-dimethyl-propylamino)-2-trifluoromethyl-benzonitrile was reacted with iodomethane to afford the title compound. MS (APCI⁺) Calc.: 270.3, Found 271.2 (M+1). ¹H NMR (CDCl₃)δ: 0.8 (d, 3H), 1.0 (d, 3H), 1.2 (d, 3H), 1.8 (bm, 1H), 2.8 (s, 3H), 3.6 (m, 1H), 6.8 (m, 1H), 6.9 (m, 1H), 7.5 (m, 1H).

### Example 25

### 2-Chloro-4-[(1-(R), 2-dimethyl-propyl)-methyl-amino]-benzonitrile

Step A: Preparation of 2-chloro-4-(1-(R), 2-dimethyl-propylamino)-benzonitrile

2-Chloro-4-fluoro-benzonitrile was reacted with 1-(R), 2-dimethyl-propylamine at 90° C. to yield the desired product.

Step B: Preparation of 2-chloro-4-[(1-(R), 2-dimethyl-propyl)-methyl-amino]-benzonitrile

2-Chloro-4-(1-(R), 2-dimethyl-propylamino)-benzonitrile was reacted with iodomethane to afford the title compound. MS (APCI⁺) Calc.: 236.8, Found: 237.2 (M+1). ¹H NMR (CDCl₃)δ: 0.8 (d, 3H), 1.0 (d, 3H), 1.2 (d, 3H), 1.8 (bm, 1H), 2.7 (s, 3H), 3.5 (m, 1H), 6.6 (m, 1H), 6.7 (s, 1H), 7.4 (m, 1H).

### Example 26

### 4-(S)-sec-Butyl-methyl-amino)-2-trifluoromethyl-benzonitrile

Step A: Preparation of 4-(S)-sec-butylamino-2-trifluoromethyl-benzonitrile

4-Fluoro-2-trifluoromethyl-benzonitrile was reacted with S-(+)-sec-butylamine to afford the desired product.

Step B: Preparation of 4-((S)-sec-butyl-methyl-amino)-2-trifluoromethyl-benzonitrile

4-(S)-sec-butylamino-2-trifluoromethyl-benzonitrile was reacted with iodomethane to afford the title compound. MS (APCI⁺) Calc.: 256.3, Found: 257.3 (M+1). ¹H NMR (CDCl₃)δ: 0.9 (t, 3H), 1.2 (d, 2H), 1.6 (m, 2H), 2.8 (s, 3H), 3.9 (m, 1H), 6.8 (m, 1H), 7.0 (m, 1H), 7.6 (m, 1H).

### Example 27

### 4-((S)-sec-Butyl-ethyl-amino)-2-trifluoromethyl-benzonitrile

4-(S)-sec-butylamino-2-trifluoromethyl-benzonitrile was reacted with iodoethane to afford the title compound. MS (APCI⁺) Calc.: 270.3, Found: 271.3 (M+1). ¹H NMR (CDCl₃)δ: 0.9 (m, 3H), 1.2 (m, 6H), 3.3 (m, 2H), 3.9 (m, 1H), 6.8 (m, 1H), 6.9 (s, 1H), 7.6 (m, 1H).

### Example 28

### 4-(2-Methyl-pyrrolidin-1-yl)-2-trifluoromethyl-benzonitrile

4- fluoro-2-trifluoromethyl-benzonitrile (0.095 g, 0.5 mmol.) and 2-methyl-pyrrolidine (0.106 g, 1.25 mmol.) were heated neat at 60° C. for 3 days. The reaction mixture was cooled and partitioned between dichloromethane and 2M HCl, dried (MgSO₄), filtered, and evaporated to dryness. The crude material was purified by preparative TLC using 30% ethyl acetate/hexanes as the eluant to afford the title compound. MS (APCI⁺) Calc.: 254.3, Found: 255.2 (M+1). ¹H NMR (CDCl₃)δ: 1.2 (m, 3H), 1.8 (m, 1H), 2.1 (m, 3H), 3.3 (m, 1H), m, 1H), 4.0 (m, 1H), 6.6 (m, 1H), 6.8 (m, 1H), 7.6 (m, 1H).

### Example 29

### 4-(Cyclopentyl-methyl-amino)-2-trifluoromethyl-benzonitrile

Following the general procedure in Example 28, 4-fluoro-2-trifluoromethyl-benzonitrile was reacted with cyclopentyl-methyl-amine at 55° C. overnight to give the desired product. The crude material was purified by preparative TLC using 20% ethyl acetate/hexanes as the eluant to afford the title compound. MS (APCI⁺) Calc.: 268.3, Found: 269.2 (M+1). ¹H NMR (CDCl₃)δ: 1.7 (bm, 8H), 2.9 (m, 3H), 4.2 (bm, 1H), 6.8 (m, 1H), 6.9 (m, 1H), (m, 1H).

### Examples 30-53

Table 1, below, provides Examples 30-53. Examples 30 through 53 can be prepared by methods analogous to the methods employed for the preparation of Examples 1-29. The compounds of Examples 30-53 can be purified and characterized according to methods analogous to the methods used for Examples 1-29.

TABLE 1

| Example | |
|---|---|
| Number | Compound |
| 30 | 4-(2-Propyl-piperidin-1-yl)-2-trifluoromethyl-benzonitrile |
| 31 | 4-(2-Ethyl-4-hydroxymethyl-piperidin-1-yl)-2-trifluoromethyl-benzonitrile |
| 32 | 4-((R)-sec-Butyl-propyl-amino)-2-trifluoromethyl-benzonitrile |
| 33 | 4-(Allyl-methyl-amino)-2-trifluoromethyl-benzonitrile |
| 34 | 4-(2-Methyl-piperidin-1-yl)-2-trifluoromethyl-benzonitrile |
| 35 | 4-(2(R)-(Ethoxy-methyl)-pyrrolidin-1-yl)-2-trifluoromethyl-benzonitrile |
| 36 | 4-(2-(2-Hydroxyethyl)-piperidin-1-yl)-2-trifluoromethyl-benzonitrile |
| 37 | 4-(2(R)-(Methoxy-methyl)-pyrrolidin-1-yl)-2-trifluoromethyl-benzonitrile |
| 38 | 4-(Ethyl-pentyl-amino)-2-trifluoromethyl-benzonitrile |
| 39 | 4-(Butyl-propyl-amino)-2-trifluoromethyl-benzonitrile |
| 40 | 4-(Isopropyl-methyl-amino)-2-trifluoromethyl-benzonitrile |
| 41 | 4-(Pentyl-propyl-amino)-2-trifluoromethyl-benzonitrile |
| 42 | 4-(2-Hydroxymethyl-piperidin-1-yl)-2-trifluoromethyl-benzonitrile |
| 43 | 4-(Butyl-ethyl-amino)-2-trifluoromethyl-benzonitrile |
| 44 | 4-(Dibutyl-amino)-2-trifluoromethyl-benzonitrile |
| 45 | 4-(2(R)-(Hydroxy-methyl)-pyrrolidin-1-yl)-2-trifluoromethyl-benzonitrile |
| 46 | 2-Chloro-4-(isopropyl-propyl-amino)-benzonitrile |
| 47 | 2-Chloro-4-(diethyl-amino)-benzonitrile |
| 48 | 2-Chloro-4-(ethyl-isopropyl-amino)-benzonitrile |
| 49 | 2-Chloro-4-(dipropyl-amino)-benzonitrile |
| 50 | 4-(Isopropyl-methyl-amino)-phthalonitrile |
| 51 | 4-(2-(R)-Methoxymethyl-pyrrolidin-1-yl)-phthalonitrile |
| 52 | 4-Azepan-1-yl-phthalonitrile |
| 53 | 2,6-Difluoro-4-(dipropyl-amino)-benzonitrile |

All salts of the Formula I compound are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

In addition, when the Formula I compound of this invention forms hydrates or solvates they are also within the scope of the invention.

The Formula I compound of this invention, and the salts thereof are all adapted to therapeutic use as agents that mediate androgen receptors in mammals, particularly humans. By virtue of this activity, these agents are useful for treating conditions that present with low bone mass and improve frailty and other disease/conditions detailed above.

The utility of the Formula I compound of the invention and the salts thereof as medical agents in the treatment of the above described disease/conditions in mammals (e.g., humans, male or female) is demonstrated by the activity of the compound of this invention in conventional assays and the in vitro and in vivo assays described below. The in vitro and in vivo assays (with appropriate modifications within the skill in the art) may be used to determine the activity of analogous agents as well as the compounds of this invention. Such assays also provide a means whereby the activities of the Formula I compound of this invention, and the salts thereof can be compared with the activities of other known compounds. The results of these comparisons are useful for determining dosage levels in mammals, including humans, for the treatment of such diseases.

The following protocols can be varied when appropriate by those skilled in the art.

### Human Androgen Receptor Binding Analysis

The following is a brief description of the assay that determines the affinity of a compound for the recombinant human androgen receptor (hAR). Competitive binding analysis is performed on baculovirus/Sf9 generated hAR extracts in the presence or absence of differing concentrations of drug and a fixed concentration of ³H-dihydrotestosterone (³H-DHT) as tracer. This binding assay method is a modification of a protocol previously described (Chang, et. al. J. Steroid Biochem. 20(1):11-17 1984). Briefly, progressively decreasing concentrations of compounds are incubated in the presence of hAR extract (Chang et al. P.N.A.S. Vol. 89, pp. 5546-5950, 1992), hydroxylapatite and 1 nM ³H-DHT for one hour at 4° C. Subsequently, the binding reactions are washed three times to completely remove excess unbound ³H-DHT. hAR bound ³H-DHT levels are determined in the presence of compounds (=competitive binding) and compared to levels bound when no competitor is present (=maximum binding). Compound binding affinity to the hAR is expressed as the concentration of compound at which one half of the maximum binding is inhibited (the IC₅₀).

Table 2, below, provides data obtained for compounds of the invention using the human androgen receptor binding analysis assay described hereinabove.

TABLE 2

| Example | AR binding |
|---|---|
| Number | IC₅₀ (nm) |
| 1 | 5.21 |
| 2 | 1.15 |
| 3 | 3.67 |
| 4 | 7.75 |
| 5 | 5.35 |
| 6 | 1.83 |
| 7 | 1.09 |
| 8 | 1.40 |
| 9 | 3.63 |
| 10 | 3.28 |
| 11 | 8.0 |
| 12 | 3.21 |
| 13 | 2.9 |
| 14 | 3.8 |
| 15 | 1.68 |
| 16 | 3.06 |
| 17 | 3.99 |
| 18 | 2.49 |
| 19 | 6.44 |
| 20 | 2.62 |
| 21 | 1.71 |
| 22 | 5.4 |
| 23 | 4.21 |
| 24 | — |
| 25 | 45.6 |
| 26 | 5.47 |
| 27 | 7.32 |
| 28 | 1.50 |
| 29 | — |
| 30 | 2.33 |
| 31 | 9.63 |
| 32 | 5.97 |
| 33 | 3.82 |
| 34 | 24.83 |
| 35 | 2.07 |
| 36 | 27.67 |
| 37 | 10.14 |
| 38 | 11.6 |
| 39 | 2.00 |
| 40 | 0.99 |
| 41 | 9.39 |
| 42 | 15.32 |
| 43 | 7.58 |
| 44 | 12.8 |
| 45 | 22.7 |
| 46 | 9.79 |
| 47 | 2.98 |
| 48 | 19.7 |
| 49 | 8.33 |
| 50 | 8.23 |
| 51 | 12.4 |
| 52 | 5.04 |
| 53 | 4.06 |

Effect of Selective Androgen Receptor Modulator on Body Weight. Body Composition and Bone Density in the Aged Intact and Ovariectomized Female Rat
The purpose of this study is to test the effects of test compound in aged intact or ovariectomized (OVX) female rat model.

### Study Protocol

Sprague-Dawley female rats were sham-operated or OVX at 18 months of age, while a group of rats was necropsied at day 0 to serve as baseline controls. One day post-surgery, the rats were treated with either vehicle or test compound. The vehicle or test compound was administered twice a week (Tuesday and Friday) by subcutaneous injection (s.c.), with the test compound being administered at an average dose of 10 milligrams per kilogram of body weight per day (10 mg/kg/day).

All rats were given s.c. injection of 10 mg/kg of calcein (Sigma, St. Louis, Mo.) for fluorescent bone label 2 and 12 days before necropsy. On the day of necropsy, all rats under ketamine/xylazine anesthesia were weighed and underwent dual-energy X-ray absorptiometry (DXA, QDR-4500/W, Hologic Inc., Waltham, Mass.) equipped with Rat Whole Body Scan software for lean and fat body mass determination. The rats were necropsied, then autopsied and blood was obtained by cardiac puncture. The distal femoral metaphysis and femoral shafts from each rat were analyzed by peripheral quantitative computerized tomography (pQCT), and volumetric total, trabecular and cortical bone mineral content and density were determined.

Peripheral Quantitative Computerized Tomography (pQCT) Analysis: Excised femurs were scanned by a pQCT X-ray machine (Stratec XCT Research M, Norland Medical Systems, Fort Atkinson, Wis.) with software version 5.40. A 1 millimeter (mm) thick cross section of the femur metaphysis was taken at 5.0 mm (proximal femoral metaphysis, a primary cancellous bone site) and 13 mm (femoral shafts, a cortical bone site) proximal from the distal end with a voxel size of 0.10 mm. Cortical bone was defined and analyzed using contour mode 2 and cortical mode 4. An outer threshold setting of 340 mg/cm³ was used to distinguish the cortical shell from soft tissue and an inner threshold of 529 mg/cm³ to distinguish cortical bone along the endocortical surface. Trabecular bone was determined using peel mode 4 with a threshold of 655 mg/cm³ to distinguish (sub)cortical from cancellous bone. An additional concentric peel of 1% of the defined cancellous bone was used to ensure that (sub)cortical bone was eliminated from the analysis. Volumetric content, density, and area were determined for both trabecular and cortical bone (Jamsa T. et al., Bone 23:155-161, 1998; Ke, H. Z. et al., Journal of Bone and Mineral Research, 16:765-773, 2001). Using the above setting, it was determined that the ex vivo precision of volumetric content, density and area of total bone, trabecular, and cortical regions ranged from 0.99% to 3.49% with repositioning.

Vaginal histology: Vaginal tissue was fixed and embedded in paraffin. Five micron sections were cut and stained with Alcian Blue staining. Histology examination of vaginal luminal epithelial thickness and mucopolysaccharide (secreted cells) was performed.

The experimental groups for the protocol are as follows:

| | |
|---|---|
| Group I: | Baseline controls |
| Group II: | Sham + Vehicle |
| Group III: | OVX + Vehicle |
| Group IV: | OVX + Test Compound at 10 mg/kg/day (in Vehicle) |

### Study Results

The preceding protocol was carried out using the compound of Example 1 as the test compound. The vehicle employed in experimental groups II-IV was 100% sesame oil. The rats in groups II-IV were dosed s.c. twice a week (on Tuesday and Friday) with 0.3 ml and 0.4 ml. This dosing provided an average daily dose of 10 mg/kg/day of the compound of Example 1 for experimental group IV. The results are provided below in Table 3.

TABLE 3

| | | | | OVX + Test |
|---|---|---|---|---|
| | Baseline | Sham | OVX | Compound |
| Body Weight (g) | 383 ± 16.3 | 434 ± 14.0^{a} | 452 ± 9.2^{a} | 459 ± 10.7^{b,c} |
| Fat Body Mass (g) | 85.0 ± 11.5 | 121.6 ± 13.9 | 148.5 ± 8.1 | 121.7 ± 7.9^{c} |
| Lean Body Mass (g) | 282 ± 10.8 | 299 ± 7.9 | 290.9 ± 6.7 | 319.0 ± 7.7^{c} |
| Total Density of | 672.7 ± 35.3 | 672.0 ± 24.3 | 593.8 ± 20.8^{a,b} | 602.2 ± 12.3^{b} |
| DFM (mg/cm³) | | | | |
| Trabecular | 351.4 ± 29.3 | 351.0 ± 24.3 | 266.7 ± 19.0^{a,b} | 310.4 ± 0.16 |
| Density of DFM | | | | |
| (mg/cm³) | | | | |
| Total Content of | 11.1 ± 0.27 | 11.9 ± 0.42 | 11.0 ± 0.21^{b} | 11.9 ± 0.16^{c} |
| FS (mg/mm) | | | | |
| Cortical Content | 11.1 ± 0.26 | 11.9 ± 0.42 | 10.9 ± 0.21^{b} | 11.8 ± 0.15^{c} |
| of FS (mg/mm) | | | | |

The values provided in Table 3 are the mean values±the standard error measurement. In Table 3 and the experimental protocol the following abbreviations or symbols had the following definitions. DFM: distal femoral metaphysis; FS: femoral shafts; g: grams; mg/cm³: milligrams per cubic centimeter; mg/mm: milligrams per millimeter; a: p<0.05 vs. Baseline; b: p<0.05 vs. Sham; c: p<0.05 vs. OVX; mg/kg/day: milligrams per kilogram of body weight per day; and ml: milliliter.

The results in Table 3 indicate that administration of the compound of Example 1 to OVX rats had negligible effect on body weight but significantly decreased fat body mass and significantly increased lean body mass when compared to OVX rats administered vehicle.

Orchiectomized Immature Rat Assay

All animal studies were performed in accordance with the Guide for Care and Use of Laboratory Animals (National Research Council, 1996). Twenty-one day old male Sprague Dawley rats were acquired from Taconic, Inc, Germantown, N.Y. Rats were housed five per cage at standard vivarium conditions (72° C., 12-h light/dark cycle), with normal chow diet and tap water ad libitum. At 25 days of age, the rats underwent bilateral orchiectomy surgery (ORX) after sedation with 3-5% Isoflurane. Following the surgery, the animals were treated immediately by daily subcutaneous (s.c.) injection with vehicle (sesame oil), testosterone propionate (TP, 10 mg/kg) and test compounds. The dosage of test compound administered is in mg/kg. At the end of the 4 days of administrations, the animals were sacrificed in a carbon dioxide chamber, the ventral prostate (VP) and levator ani muscle complex (LA) were removed and weighed. The increase in the weights of VP or LA by TP was expressed as 100%, and the ORX control as baseline, 0%; the response of the test compounds was calculated as the percentage increase compared to TP. Experimental results obtained for the compounds of Examples 1 and 7 dosed at 3, 10 and 30 mg/kg are provided in Table 4, below.

TABLE 4

| | | | | % TP | % TP |
|---|---|---|---|---|---|
| Test | Dose | | | Control | Control |
| Compound | (mg/kg) | Route | Duration | LA | VP |
| Example 1 | 3 | s.c. | 4 days | 53% | 11% |
| Example 1 | 10 | s.c. | 4 days | 93% | 30% |
| Example 1 | 30 | s.c. | 4 days | 100% | 33% |
| Example 7 | 3 | s.c. | 4 days | 40% | 23% |
| Example 7 | 10 | s.c. | 4 days | 65% | 24% |
| Example 7 | 30 | s.c. | 4 days | 89% | 39% |

Effect of Selective Androgen Receptor Modulator on Body Weight, Body Composition and Bone Density in the Aged Intact and Orchidectomized Male Rat

The purpose of this study is to test the effects of test compound in aged intact or orchidectomized (ORX) male rat model.

### Study Protocol

Male SD rats at 11 months of age were sham-operated or ORX. One day post-surgery they were treated with test compound by subcutaneous injections (s.c.) at the average dose of 3 or 10 mg/kg per day for 8 weeks. The subcutaneous injections were given 2 times (Tuesday and Friday) per week with the first injection (Tuesday) at 9 or 30 mg/kg (in sesame oil, total 0.3 ml), and the second injection (Friday) at 12 or 40 mg/kg (in sesame oil, total 0.4 ml). All rats were injected subcutaneously with calcein at 10 mg/kg on 12 and 2 days before necropsy. The experimental groups are as follows:

| | |
|---|---|
| Group I: | Sham + Vehicle |
| Group II: | Sham + Test Compound at 3 mg/kg/d |
| Group III: | Sham + Test Compound at 10 mg/kg/d |
| Group IV: | ORX + Vehicle |
| Group V: | ORX + Test Compound at 3 mg/kg/d |
| Group VI: | ORX + Test Compound at 10 mg/kg/d |

### Study Results

The preceding protocol was carried out using the compound of Example 1 as the test compound. Treatment with the compound of Example 1 according to the above protocol decreased fat body mass and increased lean body mass in both sham and ORX rats in a dose dependant manner, with the exception of Group II which showed a slight increase in fat body mass and slight decrease in lean body mass compared to Group I. Treatment with the compound of Example 1 increased trabecular density in the distal femoral metaphysis (DFM) and total density in the femoral shaft (FS) in both sham and ORX rats. Treatment with the compound of Example 1 increased levitor anni weight in both sham and ORX rats. Treatment with the compound of Example 1 did not increase prostate weight in Group II sham rats, although there was a slight increase in prostate weight of Group III compared to Group I. Treatment with the compound of Example 1 increased prostate weight in ORX rats to the level of sham controls in a dose dependent manner.

### Fracture Healing Assays

Assay for Effects on Fracture Healing After Systemic Administration

Fracture Technique: Sprage-Dawley rats at 3 months of age are anesthetized with Ketamine. A 1 cm incision is made on the anteromedial aspect of the proximal part of the right tibia or femur. The following describes the tibial surgical technique. The incision is carried through to the bone, and a 1 mm hole is drilled 4 mm proximal to the distal aspect of the tibial tuberosity 2 mm medial to the anterior ridge. Intramedullary nailing is performed with a 0.8 mm stainless steel tube (maximum load 36.3 N, maximum stiffness 61.8 N/mm, tested under the same conditions as the bones). No reaming of the medullary canal is performed. A standardized closed fracture is produced 2 mm above the tibiofibular junction by three-point bending using specially designed adjustable forceps with blunt jaws. To minimize soft tissue damage, care is taken not to displace the fracture. The skin is closed with monofilament nylon sutures. The operation is performed under sterile conditions. Radiographs of all fractures are taken immediately after nailing, and rats with fractures outside the specified diaphyseal area or with displaced nails are excluded. The remaining animals are divided randomly into the following groups with 10-12 animals per each subgroup per time point for testing the fracture healing. The first group receives daily gavage of vehicle (water: 100% Ethanol=95:5) at 1 ml/rat, while the others receive daily gavage from 0.01 to 100 mg/kg/day of the compound to be tested (1 ml/rat) for 10, 20, 40 and 80 days.

At 10, 20, 40 and 80 days, 10-12 rats from each group are anesthetized with Ketamine and sacrificed by exsanguination. Both tibiofibular bones are removed by dissection and all soft tissue is stripped. Bones from 5-6 rats for each group are stored in 70% ethanol for histological analysis, and bones from another 5-6 rats for each group are stored in a buffered Ringer's solution (+4° C., pH 7.4) for radiographs and biomechanical testing which is performed.

Histological Analysis: The methods for histologic analysis of fractured bone have been previously published by Mosekilde and Bak (The Effects of Growth Hormone on Fracture Healing in Rats: A Histological Description. Bone, 14:19-27, 1993). Briefly, the fracture site is sawed 8 mm to each side of the fracture line, embedded undecalcified in methymethacrylate, and cut frontals sections on a Reichert-Jung Polycut microtome in 8 μm thick. Masson-Trichrome stained mid-frontal sections (including both tibia and fibula) are used for visualization of the cellullar and tissue response to fracture healing with and without treatment. Sirius red stained sections are used to demonstrate the characteristics of the callus structure and to differentiate between woven bone and lamellar bone at the fracture site. The following measurements are performed: (1) fracture gap—measured as the shortest distance between the cortical bone ends in the fracture, (2) callus length and callus diameter, (3) total bone volume area of callus, (4) bony tissue per tissue area inside the callus area, (5) fibrous tissue in the callus, and (6) cartilage area in the callus.

Biomechanical Analysis: The methods for biomechanical analysis have been previously published by Bak and Andreassen (The Effects of Aging on Fracture Healing in Rats. Calcif Tissue Int 45:292-297, 1989). Briefly, radiographs of all fractures are taken prior to the biomechanical test. The mechanical properties of the healing fractures are analyzed by a destructive three- or four-point bending procedure. Maximum load, stiffness, energy at maximum load, deflection at maximum load, and maximum stress are determined.

Assay for Effects on Fracture Healing After Local Administration

Fracture Technique: Female or male beagle dogs at approximately 2 years of age are used under anesthesia in the study. Transverse radial fractures are produced by slow continuous loading in three-point bending as described by Lenehan et al. (Lenehan, T. M.; Balligand, M.; Nunamaker, D. M.; Wood, F. E.: Effects of EHDP on Fracture Healing in Dogs. J Orthop Res 3:499-507; 1985). A wire is pulled through the fracture site to ensure complete anatomical disruption of the bone. Thereafter, local delivery of prostaglandin agonists to the fracture site is achieved by slow release of compound delivered by slow release pellets or by administration of the compounds in a suitable formulation such as a paste gel solution or suspension for 10, 15, or 20 weeks.

Histological Analysis: The methods for histologic analysis of fractured bone have been previously published by Peter et al. (Peter, C. P.; Cook, W. O.; Nunamaker, D. M.; Provost, M. T.; Seedor, J. G.; Rodan, G. A. Effects of alendronate on fracture healing and bone remodeling in dogs. J. Orthop. Res. 14:74-70, 1996) and Mosekilde and Bak (The Effects of Growth Hormone on Fracture Healing in Rats: A Histological Description. Bone, 14:19-27, 1993). Briefly, after sacrifice, the fracture site is sawed 3 cm to each side of the fracture line, embedded undecalcified in methymethacrylate, and cut on a Reichert-Jung Polycut microtome in 8 μm thick of frontal sections. Masson-Trichrome stained mid-frontal sections (including both tibia and fibula) are used for visualization of the cellullar and tissue response to fracture healing with and without treatment. Sirius red stained sections are used to demonstrate the characteristics of the callus structure and to differentiate between woven bone and lamellar bone at the fracture site. The following measurements are performed: (1) fracture gap—measured as the shortest distance between the cortical bone ends in the fracture, (2) callus length and callus diameter, (3) total bone volume area of callus, (4) bony tissue per tissue area inside the callus area, (5) fibrous tissue in the callus, (6) cartilage area in the callus.

Biomechanical Analysis: The methods for biomechanical analysis have been previously published by Bak and Andreassen (The Effects of Aging on Fracture Healing in Rats. Calcif Tissue Int 45:292-297, 1989) and Peter et al. (Peter, C. P.; Cook, W. O.; Nunamaker, D. M.; Provost, M. T.; Seedor, J. G.; Rodan, G. A. Effects of Alendronate On Fracture Healing And Bone Remodeling In Dogs. J. Orthop. Res. 14:74-70, 1996). Briefly, radiographs of all fractures are taken prior to the biomechanical test. The mechanical properties of the healing fractures are analyzed by a destructive three- or four-point bending procedures. Maximum load, stiffness, energy at maximum load, deflection at maximum load, and maximum stress are determined.

1. 4-(2-Ethyl-piperidin-1-yl)-2-trifluoromethyl-benzonitrile;
4-(2-ethyl-piperidin-1-yl)-2 ,6-difluoro-benzonitrile;
2-chloro-4-(2-ethyl-piperidin-1-yl)-benzonitrile;
4-(2-ethyl-piperidin-1-yl)-phthalonitrile;
4-(sec-butyl-methyl-amino)-2-trifluoromethyl-benzonitrile;
4-(sec-butyl-ethyl-amino)-2-chloro-benzonitrile;
4-(sec-butyl-methyl-amino)-2-chloro-benzonitrile;
4-(sec-butyl-propyl-amino)-2-chloro-benzonitrile;
4-(sec-butyl-propyl-amino)-phthalonitrile;
4-(sec-butyl-ethyl-amino)-2-trifluoromethyl-benzonitrile;
4-(sec-butyl-methyl-amino)-phthalonitrile;
4-(sec-butyl-ethyl-amino)-phthalonitrile;
4-dipropylamino-2-trifluoromethyl-benzonitrile;
4-(ethyl-isopropyl-amino)-phthalonitrile;
4-dipropylamino-phthalonitrile;
4-diethylamino-2-trifluoromethyl-benzonitrile;
2-chloro-4-(ethyl-propyl-amino)-benzonitrile;
2-chloro-4-(isopropyl-methyl-amino)-benzonitrile;
4-[(1,2-dimethyl-propyl)-methyl-amino]-2-trifluoromethyl-benzonitrile;
2-chloro-4-[(1,2-dimethyl-propyl)-methyl-amino]-benzonitrile;
4-(2-methyl-pyrrolidin-1-yl)-2-trifluoromethyl-benzonitrile;
4-(cyclopentyl-methyl-amino)-2-trifluoromethyl-benzonitrile;
4-(2-propyl-piperidin-1-yl)-2-trifluoromethyl-benzonitrile;
4-(2-ethyl-4-hydroxymethyl-piperidin-1-yl)-2-trifluoromethyl-benzonitrile;
4-(sec-butyl-propyl-amino)-2-trifluoromethyl-benzonitrile;
4-(allyl-methyl-amino)-2-trifluoromethyl-benzonitrile;
4-(2-methyl-piperidin-1-yl)-2-trifluoromethyl-benzonitrile;
4-(2-(ethoxy-methyl)-pyrrolidin-1-yl)-2-trifluoromethyl-benzonitrile;
4-(2-(2-hydroxyethyl)-piperidin-1-yl)-2-trifluoromethyl-benzonitrile;
4-(2-(methoxy-methyl)-pyrrolidin-1-yl)-2-trifluoromethyl-benzonitrile;
4-(ethyl-pentyl-amino)-2-trifluoromethyl-benzonitrile;
4-(butyl-propyl-amino)-2-trifluoromethyl-benzonitrile;
4-(isopropyl-methyl-amino)-2-trifluoromethyl-benzonitrile;
4-(pentyl-propyl-amino)-2-trifluoromethyl-benzonitrile;
4-(2-hydroxymethyl-piperidin-1-yl)-2-trifluoromethyl-benzonitrile;
4-(butyl-ethyl-amino)-2-trifluoromethyl-benzonitrile;
4-(dibutyl-amino)-2-trifluoromethyl-benzonitrile;
4-(2-(hydroxy-methyl)-pyrrolidin-1-yl)-2-trifluoromethyl-benzonitrile;
2-chloro-4-(isopropyl-propyl-amino)-benzonitrile;
2-chloro-4-(diethyl-amino)-benzonitrile;
2-chloro-4-(ethyl-isopropyl-amino)-benzonitrile;
2-chloro-4-(dipropyl-amino)-benzonitrile;
4-(isopropyl-methyl-amino)-phthalonitrile;
4-(2-methoxymethyl-pyrrolidin-1-yl)-phthalonitrile;
4-azepan-1-yl-phthalonitrile; or
2,6-difluoro-4-(dipropyl-amino)-benzonitrile; or
a stereoisomer thereof or a pharmaceutically acceptable salt of said compound or stereoisomer.

2. 4-(2-(S)-Ethyl-piperidin-1-yl)-2-trifluoromethyl-benzonitrile;
4-(2-ethyl-piperidin-1-yl)-2-trifluoromethyl-benzonitrile;
4-((R)-sec-butyl-methyl-amino)-2-trifluoromethyl-benzonitrile;
4-((R)-sec-butyl-ethyl-amino)-2-trifluoromethyl-benzonitrile;
4-dipropylamino-2-trifluoromethyl-benzonitrile;
4-(sec-butyl-methyl-amino)-2-trifluoromethyl-benzonitrile;
4-diethylamino-2-trifluoromethyl-benzonitrile;
4-(sec-butyl-ethyl-amino)-2-trifluoromethyl-benzonitrile;
4-[(1(R),2-dimethyl-propyl)-methyl-amino]-2-trifluoromethyl-benzonitrile;
4-(S)-sec-butyl-methyl-amino)-2-trifluoromethyl-benzonitrile;
4-((S)-sec-butyl-ethyl-amino)-2-trifluoromethyl-benzonitrile;
4-(2-methyl-pyrrolidin-1-yl)-2-trifluoromethyl-benzonitrile;
4-(cyclopentyl-methyl-amino)-2-trifluoromethyl-benzonitrile;
4-(2-propyl-piperidin- 1-yl)-2-trifluoromethyl-benzonitrile;
4-(2-ethyl-4-hydroxymethyl-piperidin-1-yl)-2-trifluoromethyl-benzonitrile;
4-((R)-sec-butyl-propyl-amino)-2-trifluoromethyl-benzonitrile;
4-(allyl-methyl-amino)-2-trifluoromethyl-benzonitrile;
4-(2-methyl-piperidin-1-yl)-2-trifluoromethyl-benzonitrile;
4-(2-((R)-ethoxy-methyl)-pyrrolidin-1-yl)-2-trifluoromethyl-benzonitrile;
4-(2-(2-hydroxyethyl)-piperidin-1-yl)-2-trifluoromethyl-benzonitrile;
4-(2(R)-(methoxy-methyl)-pyrrolidin-1-yl)-2-trifluoromethyl-benzonitrile;
4-(ethyl-pentyl-amino)-2-trifluoromethyl-benzonitrile;
4-(butyl-propyl-amino)-2-trifluoromethyl-benzonitrile;
4-(isopropyl-methyl-amino)-2-trifluoromethyl-benzonitrile;
4-(pentyl-propyl-amino)-2-trifluoromethyl-benzonitrile;
4-(2(R)-hydroxymethyl-piperidin-1-yl)-2-trifluoromethyl-benzonitrile;
4-(butyl-ethyl-amino)-2-trifluoromethyl-benzonitrile;
4-(dibutyl-amino)-2-trifluoromethyl-benzonitrile; or
4-(2(R)-(hydroxy-methyl)-pyrrolidin-1-yl)-2-trifluoromethyl-benzonitrile; or
a pharmaceutically acceptable salt of said compound.

3. 4-(2-(S)-Ethyl-piperidin-1-yl)-2-trifluoromethyl-benzonitrile or
4-((R)-sec-butyl-methyl-amino)-2-trifluoromethyl-benzonitrile, or
a pharmaceutically acceptable salt thereof.

4. 2-Chloro-4-(2-(S)-ethyl-piperidin-1-yl)-benzonitrile;
4-((R)-sec-butyl-ethyl-amino)-2-chloro-benzonitrile;
4-((R)-sec-butyl-methyl-amino)-2-chloro-benzonitrile;
4-((R)-sec-butyl-propyl-amino)-2-chloro-benzonitrile;
2-chloro-4-(ethyl-propyl-amino)-benzonitrile;
2-chloro-4-(isopropyl-methyl-amino)-benzonitrile;
2-chloro-4-[(1(R),2-dimethyl-propyl)-methyl-amino]-benzonitrile;
2-chloro-4-(isopropyl-propyl-amino)-benzonitrile;
2-chloro-4-(diethyl-amino)-benzonitrile;
2-chloro-4-(ethyl-isopropyl-amino)-benzonitrile; or
2-chloro-4-(dipropyl-amino)-benzonitrile; or
a pharmaceutically acceptable salt of said compound.

5. 4-(2-(S)-Ethyl-piperidin-1-yl)-phthalonitrile;
4-(2-ethyl-piperidin-1-yl)-phthalonitrile;
4-((R)-sec-butyl-propyl-amino)-phthalonitrile;
4-((R)-sec-butyl-methyl-amino)-phthalonitrile;
4-(R)-sec-butyl-ethyl-amino)-phthalonitrile;
4-(ethyl-isopropyl-amino)-phthalonitrile;
4-(sec-butyl-propyl-amino)-phthalonitrile;
4-dipropylamino-phthalonitrile;
4-(isopropyl-methyl-amino)-phthalonitrile;
4-(2-(R)-methoxymethyl-pyrrolidin-1-yl)-phthalonitrile; or
4-azepan-1-yl-phthalonitrile; or
a pharmaceutically acceptable salt of said compound.

6. 4-(2-Ethyl-piperidin-1-yl)-2,6-difluoro-benzonitrile; or
2,6-difluoro-4-(dipropyl-amino)-benzonitrile; or
a pharmaceutically acceptable salt of said compound.

7. A pharmaceutical composition comprising a therapeutically effective amount of a compound of claim 1, or a pharmaceutically acceptable salt of said compound and a pharmaceutically acceptable vehicle, diluent or carrier.

8. A pharmaceutical composition of claim 7 wherein said compound is
4-(2-(S)-ethyl-piperidin-1-yl)-2-trifluoromethyl-benzonitrile or
4-((R)-sec-butyl-methyl-amino)-2-trifluoromethyl-benzonitrile, or
a pharmaceutically acceptable salt thereof.

9. A method of treating a condition that presents with low bone mass, treating a wasting disease, increasing muscle mass, increasing lean body mass, decreasing fat body mass or treating bone fracture and muscle damage in a mammal, the method comprising administering to said mammal a therapeutically effective amount of a compound of Formula I 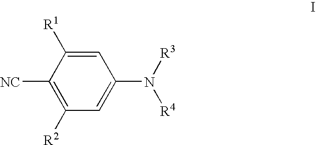
a prodrug thereof or a pharmaceutically acceptable salt of said compound or said prodrug, wherein:
R¹ is hydrogen;
R² is chloro, cyano or trifluoromethyl;
or R¹ and R² are each fluoro;
R³ and R⁴ are each independently (C₁-C₆)alkyl, (C₃-C₇)cycloalkyl or (C₂-C₆)alkenyl; or R³ and R⁴ taken together with the nitrogen to which they are attached is 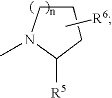
is 1, 2 or 3;
R⁵ is (C₁-C₆)alkyl optionally substituted with hydroxy or (C₁-C₆)alkoxy; and
R⁶ is hydrogen or (C₁-C₆)alkyl optionally substituted with hydroxy or (C₁-C₆)alkoxy.

10. The method of claim 9 wherein the compound of Formula I is:
4-(2-ethyl-piperidin-1-yl)-2-trifluoromethyl-benzonitrile;
4-(2-ethyl-piperidin-1-yl)-2,6-difluoro-benzonitrile;
2-chloro-4-(2-ethyl-piperidin-1-yl)-benzonitrile;
4-(2-ethyl-piperidin-1-yl)-phthalonitrile;
4-(sec-butyl-methyl-amino)-2-trifluoromethyl-benzonitrile;
4-(sec-butyl-ethyl-amino)-2-chloro-benzonitrile;
4-(sec-butyl-methyl-amino)-2-chloro-benzonitrile;
4-(sec-butyl-propyl-amino)-2-chloro-benzonitrile;
4-(sec-butyl-propyl-amino)-phthalonitrile;
4-(sec-butyl-ethyl-amino)-2-trifluoromethyl-benzonitrile;
4-(sec-butyl-methyl-amino)-phthalonitrile;
4-(sec-butyl-ethyl-amino)-phthalonitrile;
4-dipropylamino-2-trifluoromethyl-benzonitrile;
4-(ethyl-isopropyl-amino)-phthalonitrile;
4-dipropylamino-phthalonitrile;
4-diethylamino-2-trifluoromethyl-benzonitrile;
2-chloro-4-(ethyl-propyl-amino)-benzonitrile;
2-chloro-4-(isopropyl-methyl-amino)-benzonitrile;
4-[(1,2-dimethyl-propyl)-methyl-amino]-2-trifluoromethyl-benzonitrile;
2-chloro-4-[(1,2-dimethyl-propyl)-methyl-amino]-benzonitrile;
4-(2-methyl-pyrrolidin-1-yl)-2-trifluoromethyl-benzonitrile; and
4-(cyclopentyl-methyl-amino)-2-trifluoromethyl-benzonitrile;
4-(2-propyl-piperidin-1-yl)-2-trifluoromethyl-benzonitrile;
4-(2-ethyl-4-hydroxymethyl-piperidin-1-yl)-2-trifluoromethyl-benzonitrile;
4-(sec-butyl-propyl-amino)-2-trifluoromethyl-benzonitrile;
4-(allyl-methyl-amino)-2-trifluoromethyl-benzonitrile;
4-(2-methyl-piperidin-1-yl)-2-trifluoromethyl-benzonitrile;
4-(2-(ethoxy-methyl)-pyrrolidin-1-yl)-2-trifluoromethyl-benzonitrile;
4-(2-(2-hydroxyethyl)-piperidin-1-yl)-2-trifluoromethyl-benzonitrile;
4-(2-(methoxy-methyl)-pyrrolidin-1-yl)-2-trifluoromethyl-benzonitrile;
4-(ethyl-pentyl-amino)-2-trifluoromethyl-benzonitrile;
4-(butyl-propyl-amino)-2-trifluoromethyl-benzonitrile;
4-(isopropyl-methyl-amino)-2-trifluoromethyl-benzonitrile;
4-(pentyl-propyl-amino)-2-trifluoromethyl-benzonitrile;
4-(2-hydroxymethyl-piperidin-1-yl)-2-trifluoromethyl-benzonitrile;
4-(butyl-ethyl-amino)-2-trifluoromethyl-benzonitrile;
4-(dibutyl-amino)-2-trifluoromethyl-benzonitrile;
4-(2-(hydroxy-methyl)-pyrrolidin-1-yl)-2-trifluoromethyl-benzonitrile;
2-chloro-4-(isopropyl-propyl-amino)-benzonitrile;
2-chloro-4-(diethyl-amino)-benzonitrile;
2-chloro-4-(ethyl-isopropyl-amino)-benzonitrile;
2-chloro-4-(dipropyl-amino)-benzonitrile;
4-(isopropyl-methyl-amino)-phthalonitrile;
4-(2-methoxymethyl-pyrrolidin-1-yl)-phthalonitrile;
4-azepan-1-yl-phthalonitrile; or
2,6-difluoro-4-(di propyl-amino)-benzonitrile; or
a stereoisomer thereof or a pharmaceutically acceptable salt of said compound or stereoisomer.

11. The method of claim 10 wherein the condition presents with low bone mass.

12. The method of claim 11 wherein the condition is osteoporosis, a bone defect, childhood idiopathic bone loss, alveolar bone loss, mandibular bone loss, bone fracture, osteotomy, periodontitis or prosthetic ingrowth.

13. The method of claim 10 wherein concomitant bone fracture and muscle damage are treated.

14. The method of claim 10 wherein muscle mass is increased, lean body mass is increased or fat body mass is decreased.

15. The method of claim 14 wherein the mammal is a livestock mammal.

16. The method of claim 9 wherein the therapeutically effective amount of the compound of Formula I is about 0.01 mg/kg/day to about 10 mg/kg/day.
